(19) United States
(12) Patent Application Publication
(10) Pub. No.: US 2010/0135916 A1
(43) Pub. Date: Jun. 3, 2010

(54) Water in silicone emulsions containing elastomers

(76) Inventors: Benedicte Courel, Waterloo (BE); Katrin Steinbach, Nivelles (BE)

Correspondence Address: DOW CORNING CORPORATION CO1232, 2200 W. SALZBURG ROAD, P.O. BOX 994, MIDLAND, MI, 48686-0994, US

(21) Appl. No.: 11/886,173

(22) PCT Filed: Mar. 21, 2006
(86) PCT No.: PCT/US2006/010185

§ 371 Date: Sep. 10, 2007

Related U.S. Application Data
(60) Provisional application No. 60/669,969, filed on Apr. 7, 2005.

(51) Int. Cl. A61K 9/12; A61Q 17/04; A61Q 19/04; A61Q 1/00

(52) U.S. Cl. 424/47; 424/45

(57) ABSTRACT

Water in silicone emulsions, methods for preparing the emulsions, and personal care products containing the emulsions are disclosed. The silicone phase of the water in silicone emulsions contains an emollient, a silicone elastomer containing a hydrophilic group, and an optional nonionic surfactant. The aqueous phase contains an anionic surfactant at such a concentration to provide a weight ratio of the silicone elastomer to anionic surfactant ranging from 60/1 to 1/1 in the water in silicone emulsion. The resulting water in silicone emulsions can be used in a variety of personal care, household and health care formulations, and in particular for sprayable lotions.

### CROSS REFERENCE TO RELATED APPLICATIONS

This application claims priority to U.S. Provisional Application No. 60/669,969, filed Apr. 7, 2005.

### TECHNICAL FIELD

This invention relates to water in silicone emulsions, methods for preparing the emulsions, and personal care products containing the emulsions. The silicone phase of the water in silicone emulsions contains an emollient, a silicone elastomer containing a hydrophilic group, and an optional nonionic surfactant. The aqueous phase contains an anionic surfactant at such a concentration to provide a weight ratio of the silicone elastomer to anionic surfactant ranging from 60/1 to 1/1 in the water in silicone emulsion. The resulting water in silicone emulsions can be used in a variety of personal care, household and health care formulations, and in particular for sprayable lotions.

### BACKGROUND

Water in silicone emulsions are used as a base formulation for many personal care and cosmetic product compositions. Many of these compositions are formulated using a high molecular weight silicone polyether as the primary emulsifier. More recently, silicone elastomers have been incorporated into the continuous silicone phase of such formulations to provide enhanced aesthetic benefits and/or additional functional performance. However, the incorporation of higher molecular weight silicone elastomers makes it difficult to formulate water in silicone emulsions having lower viscosities, and in particular, storage stable water in silicone based emulsions that are sprayable.

The present inventors have discovered that certain combinations of polyether functional silicone elastomers and anionic surfactants provide stable water in silicone emulsions. Furthermore, the present invention provides stable low viscosity water in silicone emulsions containing silicone elastomers that are sprayable.

### SUMMARY

This invention relates to a water in silicone emulsion comprising:

a silicone phase containing;
A) an emollient,
B) a silicone elastomer containing a hydrophilic group,
C) an optional organic non-ionic surfactant, and an aqueous phase containing;
D) an anionic surfactant,
   wherein the weight ratio of the polyether functional silicone elastomer B) to the anionic surfactant D) ranges from 60/1 to 1/1.

This invention further provides a method for making a water in silicone emulsion comprising:

I) preparing a silicone phase comprising;
A) an emollient,
B) a silicone elastomer containing a hydrophilic group,
C) an optional organic non-ionic surfactant, and an aqueous phase containing;
D) an anionic surfactant,
   wherein the weight ratio of the polyether functional silicone elastomer B) to the anionic surfactant D) ranges from 60/1 to 1/1,

II) adding the aqueous phase to the silicone phase with mixing.

This invention further relates to personal care products containing the present water in silicone emulsions.

### DETAILED DESCRIPTION

### A) The Emollient

Component (A) in the present invention is an emollient. As used herein, “emollient” encompasses any material, or combination of materials, known in the art as an emollient. Typically the emollient (A) is an organic oil or a silicone. Typically, the emollient contains at least 0.1 weight percent alternatively 1 to 5, or alternatively 5 to 60 wt % of a low molecular weight siloxane. As used herein, the phrase low molecular weight siloxane is intended to mean and to include polysiloxanes having the general formula RᵢSiO_{(4-i)/2} in which i has an average value of one to three, and having a molecular weight (M_{w}) of less than 1000, R is any monovalent organic group, but typically R is a methyl group. Alternatively, the structures of the low molecular weight siloxanes can be represented by monofunctional “M” units (CH₃)₃SiO_{1/2}, difunctional “D” units (CH₃)₂SiO₂/, trifunctional “T” units, and tetrafunctional “Q” units SiO_{4/2}. The phrase “low molecular weight siloxane” is intended to mean and to include (i) low molecular weight linear and cyclic volatile methyl siloxanes, (ii) low molecular weight functional linear and cyclic siloxanes. Most preferred, however, are the low molecular weight linear and cyclic volatile methyl siloxanes (VMS). Volatile methyl siloxanes conforming to the CTFA definition of cyclomethicones are also considered to be within the definition of low molecular weight siloxane.

Linear VMS have the formula (CH₃)₃SiO{(CH₃)₂SiO}_{f}Si(CH₃)₃. The value off is 0-7. Cyclic VMS have the formula {(CH₃)₂SiO}_{g}. The value of g is 3-6. Preferably, these volatile methyl siloxanes have a molecular weight of less than about 1,000; a boiling point less than about 250° C.; and a viscosity of about 0.65 to about 5.0 centistoke (mm²/s), generally not greater than 5.0 centistoke (mm²/s).

Representative linear volatile methyl siloxanes are hexamethyldisiloxane (MM) with a boiling point of 100° C., viscosity of 0.65 mm²/s, and formula Me₃SiOSiMe₃; octamethyltrisiloxane (MDM) with a boiling point of 152° C., viscosity of 1.04 mm²/s, and formula Me₃SiOMe₂SiOSiMe₃; decamethyltetrasiloxane (MD₂M) with a boiling point of 194° C., viscosity of 1.53 mm²/s, and formula Me₃SiO(Me₂SiO)₂SiMe₃; dodecamethylpentasiloxane (MD₃M) with a boiling point of 229° C., viscosity of 2.06 mm²/s, and formula Me₃SiO(Me₂SiO)₃SiMe₃; tetradecamethylhexasiloxane (MD₄M) with a boiling point of 245° C., viscosity of 2.63 mm²/s, and formula Me₃SiO(Me₂SiO)₄SiMe₃; and hexadecamethylheptasiloxane (MD₅M) with a boiling point of 270° C., viscosity of 3.24 mm²/s, and formula Me₃SiO(Me₂SiO)₅SiMe₃.

Representative cyclic volatile methyl siloxanes are hexamethylcyclotrisiloxane (D₃), a solid with a boiling point of 134° C., a molecular weight of about 223, and formula {(Me₂)SiO}₃; octamethylcyclotetrasiloxane (D₄) with a boiling point of 176° C., viscosity of 2.3 mm²/s, a molecular weight of about 297, and formula {(Me₂)SiO}₄; decamethylcyclopentasiloxane (D₅) with a boiling point of 210° C., viscosity of 3.87 mm²/s, a molecular weight of about 371, and formula {(Me₂)SiO}₅; and dodecamethylcyclohexasiloxane (D₆) with a boiling point of 245° C., viscosity of 6.62 mm²/s, a molecular weight of about 445, and formula {(Me₂)SiO}₆.

Representative branched volatile methyl siloxanes are heptamethyl-3-{(trimethylsilyl)oxy}trisiloxane (M₃T) with a boiling point of 192° C., viscosity of 1.57 mm²/s, and formula C₁₀H₃₀O₃Si₄; hexamethyl-3,3,bis{(trimethylsilyl)oxy}trisiloxane (M₄Q) with a boiling point of 222° C., viscosity of 2.86 mm²/s, and formula C₁₂H₃₆O₄Si₅; and pentamethyl {(trimethylsilyl)oxy}cyclotrisiloxane (MD₃) with the formula C₈H₂₄O₄Si₄.

The invention also includes using low molecular weight linear and cyclic volatile and non-volatile higher alkyl and aryl siloxanes, represented respectively by formulas R₃SiO(R₂SiO)_{f}SiR₃ and (R₂SiO)_{g}. R can be alkyl groups with 2-20 carbon atoms or aryl groups such as phenyl. The value of f is 0 to about 7. The value of g is 3-6. These values should be selected to provide polysiloxanes with a viscosity generally not greater than about 5 centistoke (mm²/s), and with a molecular weight of less than about 1,000. Illustrative of such polysiloxanes are polydiethylsiloxane, polymethylethylsiloxane, polymethylphenylsiloxane, and polydiphenylsiloxane.

Low molecular weight functional polysiloxanes can also be employed, and are represented by the formula R₃SiO(RQSiO)_{f}SiR₃ or the formula (RQSiO)_{g} where Q is a functional group. Examples of such functional polysiloxanes are acrylamide functional siloxane fluids, acrylate functional siloxane fluids, amide functional siloxane fluids, amino functional siloxane fluids, carbinol functional siloxane fluids, carboxy functional siloxane fluids, chloroalkyl functional siloxane fluids, epoxy functional siloxane fluids, glycol functional siloxane fluids, ketal functional siloxane fluids, mercapto functional siloxane fluids, methyl ester functional siloxane fluids, perfluoro functional siloxane fluids, silanol functional siloxanes, and vinyl functional siloxane fluids. Again, the values off and g, and the functional group Q, are selected to provide functional polysiloxanes with a viscosity generally not greater than about 5 centistoke (mm²/s), and a molecular weight of less than about 1,000.

The emollient A) may contain other components that are dispersible in the other selected components. These other components can be selected from any silicone, organic, or personal care active that is substantially soluble in the low molecular weight siloxane, and conversely, is substantially insoluble in water. Thus, other typical emollient components can include, high molecular weight (i.e. M_{w}>1000) siloxanes, including silicone elastomers and resins, hydrocarbon oils, waxes, emollients, fragrances, and personal care organic actives such as vitamins and sunscreens. Typically, when the other emollient component is primarily a polar oil such as a vegetable oil, it is preferred that at least 50 weight percent of the emollient contain a low molecular weight siloxane.

High molecular weight siloxanes can be added to the emollient having the formula RiSiO_{(4-i)/2} in which i has an average value of one to three, R is a monovalent organic group. The phrase high molecular weight means a molecular weight (M_{w}) of equal to or greater than 1000. Thus, the high molecular weight siloxane can be selected from any polydiorganosiloxanes fluids or gum having a molecular weight equal to or greater than 1000. The polydiorganosiloxane gums suitable for the present invention are essentially composed of dimethylsiloxane units with the other units being represented by monomethylsiloxane, trimethylsiloxane, methylvinylsiloxane, methylethylsiloxane, diethylsiloxane, methylphenylsiloxane, diphenylsiloxane, ethylphenylsiloxane, vinylethylsiloxane, phenylvinylsiloxane, 3,3,3-trifluoropropylmethylsiloxane, dimethylphenylsiloxane, methylphenylvinylsiloxane, dimethylethylsiloxane, 3,3,3-trifluoropropyldimethylsiloxane, mono-3,3,3-trifluoropropylsiloxane, aminoalkylsiloxane, monophenylsiloxane, monovinylsiloxane and the like. The polydiorganosiloxane gums are well known in the art and can be obtained commercially, and which have viscosities greater than 1,000,000 cs. at 25.degree. C., preferably greater than 5,000,000 cs. at 25.degree. C.

Suitable oil components include, but are not limited to, natural oils such as coconut oil; hydrocarbons such as mineral oil and hydrogenated polyisobutene; fatty alcohols such as octyldodecanol; esters such as C12-C15 alkyl benzoate; diesters such as propylene dipelarganate; and triesters, such as glyceryl trioctanoate. The other emollient components can also be mixture of low viscosity and high viscosity oils. Suitable low viscosity oils have a viscosity of 5 to 100 mPa·s at 25° C., and are generally esters having the structure RCO-OR′ wherein RCO represents the carboxylic acid radical and wherein OR′ is an alcohol residue. Examples of these low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or mixtures of octyldodecanol, acetylated lanolin alcohol, cetyl acetate, isododecanol, polyglyceryl-3-diisostearate, or mixtures thereof. The high viscosity surface oils generally have a viscosity of 200-1,000,000 mPa·s at 25° C., preferably a viscosity of 100,000-250,000 mPa·s. Surface oils include castor oil, lanolin and lanolin derivatives, triisocetyl citrate, sorbitan sesquioleate, C10-18 triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or mixtures thereof. Mention may be made, among the optional other non-silicone fatty substances, of mineral oils, such as liquid paraffin or liquid petroleum, of animal oils, such as perhydrosqualene or arara oil, or alternatively of vegatable oils, such as sweet almond, calophyllum, palm, castor, avocado, jojaba, olive or cereal germ oil. It is also possible to use esters of lanolic acid, of oleic acid, of lauric acid, of stearic acid or of myristic acid, for example; alcohols, such as oleyl alcohol, linoleyl or linolenyl alcohol, isostearyl alcohol or octyldodecanol; or acetylglycerides, octanoates, decanoates or ricinoleates of alcohols or of polyalcohols. It is alternatively possible to use hydrogenated oils which are solid at 25° C., such as hydrogenated castor, palm or coconut oils, or hydrogenated tallow; mono-, di-, tri- or sucroglycerides; lanolins; or fatty esters which are solid at 25° C.

The common assignee's U.S. Pat. No. 5,948,855 (Sep. 7, 1999), also contains an extensive list of some appropriate oil and water soluble active ingredients such as vitamins, sunscreens, anti-ageing actives, fragrances, essential oils, preservatives, ceramides, amino-acid derivatives, liposomes, botanicals (plant extracts) and additional conditioning agents such as glycols, glycerine, quaternary polymers. Other additives can include, depending on the use, vitamins A and E in their various forms, including but not limited to, Vitamin A₁, RETINOL, C₂-C₁₈ esters of RETINOL, Vitamin E, TOCOPHEROL, esters of Vitamin E, and mixtures thereof. RETINOL includes trans-RETINOL, 13-cis-RETINOL, 11-cis-RETINOL, 9-cis-RETINOL, and 3,4-didehydro-RETINOL. Other vitamins which are appropriate include RETINYL ACETATE, RETINYL PALMITATE, RETINYL PROPIONATE, α-TOCOPHEROL, TOCOPHERSOLAN, TOCOPHERYL ACETATE, TOCOPHERYL LINOLEATE, TOCOPHERYL NICOTINATE, and TOCOPHERYL SUCCINATE, sunscreen agents, humectants, preservatives, such as known parabens, emollients, occlusive agents, and esters. Other additives can include pigments especially when the emulsion is used as a make-up. The compositions according to the invention can also contain agents for artificially tanning and/or browning the skin (self-tanning or bronzing agents), such as, and for example, dihydroxyacetone (DHA) or erythrulose and drugs which can be used either in the oil/silicone phase or water phase of the water in silicone emulsions.

Pigments can be added to the emollient component. Typical pigments are iron oxides and titanium dioxide which can be present in the composition in the amount of 0.1 to 30 wt.-%, preferably 5 to 20 wt.-% and most preferably 8 to 14 wt.-%.

Powders can be added to the emollient component. The powder component of the invention can be generally defined as dry, particulate matter having a particle size of 0.02-50 microns. The particulate matter may be colored or non-colored (for example white). Suitable powders include bismuth oxychloride, titanated mica, fumed silica, spherical silica beads, polymethylmethacrylate beads, micronized teflon, boron nitride, acrylate polymers, aluminum silicate, aluminum starch octenylsuccinate, bentonite, calcium silicate, cellulose, chalk, corn starch, distomaceous earth, fuller's earth, glyceryl starch, hectorite, hydrated silica, kaolin, magnesium aluminum silicate, magnesium carbonate, magnesium hydroxide, magnesium oxide, magnesium silicate, magnesium trisilicate, maltodextrin, montmorillonite, microcrystalline cellulose, rice starch, silica, talc, mica, titanium dioxide, zinc laurate, zinc myristate, zinc neodecanoate, zinc rosinate, zinc stearate, polyethylene, alumina, attapulgite, calcium carbonate, calcium silicate, dextran, kaolin, nylon, silica silylate, silk powder, serecite, soy flour, tin oxide, titanium hydroxide, trimagnesium phosphate, walnut shell powder, or mixtures thereof. The above mentioned powders may be surface treated with lecithin, amino acids, mineral oil, silicone oil, or various other agents either alone or in combination, which coat the powder surface and render the particles hydrophobic in nature. The powder component also comprises various organic and inorganic pigments. The organic pigments are generally various aromatic types including azo, indigoid, triphenylmethane, anthraquinone, and xanthine dyes which are designated as D&C and FD&C blues, browns, greens, oranges, reds, yellows, etc. Inorganic pigments generally consist of insoluble metallic salts of certified color additives, referred to as the Lakes or iron oxides.

A pulverulent colouring agent, such as carbon black, chromium or iron oxides, ultramarines, manganese pyrophosphate, iron blue, and titanium dioxide, pearlescent agents, generally used as a mixture with coloured pigments, or some organic dyes, generally used as a mixture with coloured pigments and commonly used in the cosmetics industry, can be added to the composition. In general, these coulouring agents can be present in an amount by weight from 0 to 20% with respect to the weight of the final composition.

Pulverulent inorganic or organic fillers can also be added, generally in an amount by weight from 0 to 40% with respect to the weight of the final composition. These pulverulent fillers can be chosen from talc, micas, kaolin, zinc or titanium oxides, calcium or magnesium carbonates, silica, spherical titanium dioxide, glass or ceramic beads, metal soaps derived from carboxylic acids having 8-22 carbon atoms, non-expanded synthetic polymer powders, expanded powders and powders from natural organic compounds, such as cereal starches, which may or may not be crosslinked.

Fillers may be present in a proportion of from 0 to 35% of the total weight of the composition, more preferably 5 to 15%. Mention may be made in particular of talc, mica, silica, kaolin, nylon powders (in particular ORGASOL), polyethylene powders, Teflon, starch, boron nitride, copolymer microspheres such as EXPANCEL (Nobel Industrie), polytrap and silicone resin microbeads (TOSPEARL from Toshiba, for example).

The composition according to the invention can include a sunscreen. Sunscreens include those components which absorb ultraviolet light between 290 and 320 nanometers, i.e., the UV-B region, such as para-aminobenzoic acid derivatives and cinnamates such as octyl methoxy cinnamate; and those compositions which absorb ultraviolet light in the range of 320 to 400 nanometer, i.e., the UV-A region, such as benzophenone and butyl methoxy dibenzoylmethane, and hydrophilic compositions such as benzylidine-2-camphor sulphonic acid derivatives.

The compositions of the present invention may contain 1 to 95 wt %, alternatively 5 to 90 wt %, or alternatively 10 to 80 weight % of the emollient, component (A).

### B) The Silicone Elastomer Containing a Hydrophilic Group

Component B) in the present invention a silicone elastomer containing a hydrophilic group. For purposes of this invention, “hydrophilic group” is the accepted meaning in the art, i.e, designating water loving chemical moieties. Thus, the hydrophilic group can be selected from various cationic, anionic, zwitterionic, polyoxyalkylene, oxoazoline chemical moieties that are commonly used in combination with various hydrophobic chemical moieties to create surfactant structures or molecules having surface-active behavior. The amount of the hydrophilic substituent on the organopolysiloxane can vary, depending on the specific chemical component, providing there is at least one hydrophilic group present on the organopolysiloxane.

Any silicone elastomer containing a polyether functional organic group may be used as component B). Alternatively, the polyether functional silicone elastomer selected as component B) may be prepared by a first step of reacting (a) ≡Si—H an containing polysiloxane; and (b) a mono-alkenyl polyether; in the presence of a platinum catalyst, until an ≡Si—H containing siloxane with polyether groups is formed, or alternatively, reacting (c) the ≡Si—H containing siloxane with polyether groups; and (d) an unsaturated hydrocarbon such as an alpha, omega-diene; in the presence of (e) a solvent and a platinum catalyst, until a silicone elastomer is formed by crosslinking and addition of ≡SiH across double bonds in the alpha, omega-diene, as taught in U.S. Pat. No. 5,811,487, which is hereby incorporated by reference in its entirety.

A representative, non-limiting example of a polyether functional silicone elastomer suitable as component (B) is Dow Corning® 9011 Silicone Elastomer Blend (Dow Corning Corporation, Midland, Mich.).

The emulsion compositions of the present invention may contain 0.1 to 50 wt %, alternatively 0.5 to 35 wt %, or alternatively 1 to 25 weight % of the, component (B).

### C) The Optional Nonionic Surfactant

Optional component C) is a non-ionic surfactant. Any nonionic surfactant known in the art to stabilize emulsions, and in particular water in silicone emulsions, may be used as component C). The optional non-ionic surfactant is added to the silicone phase, when preparing the emulsion. Typically, component C) is an alkoxylated alcohol, alternatively an alkoxylated fatty alcohol, or alternatively a propyloxylated fatty alcohol. When component C) is a propyloxylated fatty alcohol, the number of propylene oxide units present in the molecule can vary from 1 to 30, alternatively, 1 to 10, or alternatively from 1 to 5 propylene oxide units, and the number of carbon atoms in the fatty alcohol can vary from 8 to 30, alternatively from 12 to 20, or alternatively from 12 to 18 carbon atoms. Alternatively, component C) may be a propyloxlated myristyl alcohol (i.e. a polypropylene glycol ether of myristyl alcohol), containing 1 to 6, alternatively 3 propylene oxide units. Representative, non-limiting examples of a propyloxylated myristyl alcohol containing 3 propylene oxide units include the following products sold commercially under the following tradenames;

Acconon MA 3(Abitec Corporation),

Jeecol PMA-3(Jeen International Corporation),

Procol PMA-3(Protameen Chemicals, Inc.),

Promyristyl PM3(Croda Chemicals Europe, Ltd.),

Promyristyl PM-3(Croda, Inc.),

Varonic APM(Degussa Care Specialties).

The emulsion compositions of the present invention may contain 0 to 30 wt %, alternatively 0.1 to 20 wt %, or alternatively 0.1 to 5 weight % of the component (C).

### D) The Anionic Surfactant

Component (D) is an anionic surfactant. Any anionic surfactant known in the art to stabilize emulsions may be selected as component (D). Examples of suitable anionic surfactants include alkali metal sulforicinates, sulfonated glyceryl esters of fatty acids such as sulfonated monoglycerides of coconut oil acids, salts of sulfonated monovalent alcohol esters such as sodium oleylisothianate, amides of amino sulfonic acids such as the sodium salt of oleyl methyl tauride, sulfonated products of fatty acids nitriles such as palmitonitrile sulfonate, sulfonated aromatic hydrocarbons such as sodium alpha-naphthalene monosulfonate, condensation products of naphthalene sulfonic acids with formaldehyde, sodium octahydroanthracene sulfonate, alkali metal alkyl sulfates such as sodium lauryl sulfate, ammonium lauryl sulfate or triethanol amine lauryl sulfate, ether sulfates having alkyl groups of 8 or more carbon atoms such as sodium lauryl ether sulfate, ammonium lauryl ether sulfate, sodium alkyl aryl ether sulfates, and ammonium alkyl aryl ether sulfates, alkylarylsulfonates having 1 or more alkyl groups of 8 or more carbon atoms, alkylbenzenesulfonic acid alkali metal salts exemplified by hexylbenzenesulfonic acid sodium salt, octylbenzenesulfonic acid sodium salt, decylbenzenesulfonic acid sodium salt, dodecylbenzenesulfonic acid sodium salt, cetylbenzenesulfonic acid sodium salt, and myristylbenzenesulfonic acid sodium salt, sulfuric esters of polyoxyethylene alkyl ether including CH₃(CH₂)₆CH₂O(C₂H₄O)₂SO₃H, CH₃(CH₂)₇CH₂O(C₂H₄O)_{3.5}SO₃H, CH₃(CH₂)₈CH₂O(C₂H₄O)₈SO₃H, CH₃(CH₂)₁₉CH₂O(C₂H₄O)₄SO₃H, and CH₃(CH₂)₁₀CH₂O(C₂H₄O)₆SO₃H, sodium salts, potassium salts, and amine salts of alkylnaphthylsulfonic acid.

Alternatively, the anionic surfactant is selected from a sulfate of ethoxylated alcohols. Sulfates of ethoxylated alcohols are well known in the art and many are sold commercially under numerous tradenames such as ALFONINIC, NEODOL, STANDAPOL ES, STEOL, SULFOTEX, TEXAPON, WICOLATE. Alternatively, the anionic surfactant is STANDAPOL ES or EMPICOL ESB-3.

The compositions of the present invention contain 0.01 to 30 wt %, alternatively 0.1 to 20 wt %, or alternatively 0.1 to 5 weight % of the anionic surfactant, component (D).

The water in silicone emulsions are stabilized by the combination of components B) and D). As such, the water in silicone emulsion compositions contain concentrations of the polyether functional silicone elastomer B) and the anionic surfactant D) such that the weight ratio of the weight ratio of the polyether functional silicone elastomer B) to the anionic surfactant D) ranges from 60/1 to 1/1, alternatively from 40/1 to 10/1, or alternatively 20/1 to 1/1.

The water in silicone emulsions of the present invention can be prepared by any method known in the art for producing water in silicone emulsions, or alternatively, can be prepared according to the present inventive methods, discussed below.

This invention further provides a method for making a water in silicone emulsion comprising:

I) preparing a silicone phase comprising;
A) an emollient,
B) a silicone elastomer containing a hydrophilic group,
C) an optional organic non-ionic surfactant, and an aqueous phase containing;
D) an anionic surfactant,
   wherein the weight ratio of the polyether functional silicone elastomer B) to the anionic surfactant D) ranges from 60/1 to 1/1,

II) adding the aqueous phase to the silicone phase with mixing.

Components A), B), C), and D) are the same as described above. The present method involves preparing a silicone phase containing components A), B) and optionally C), and an aqueous phase containing the anionic surfactant D). Both the silicone phase and aqueous phase are prepared by simply combining the components, typically with simple mixing. The aqueous phase is then added to the silicone phase with mixing. There are no special requirements or conditions needed to effect the mixing. Mixing techniques can be simple stirring, homogenizing, sonalating, and other mixing techniques known in the art to effect the formation of emulsions, in particular water in silicone emulsions. The mixing can be conducted in a batch, semi-continuous, or continuous process.

The water in silicone emulsions prepared according to the invention can be used in various over-the-counter (OTC) personal care compositions, health care compositions, and household care compositions, but especially in the personal care arena. The compositions prepared according to the present invention can be combined with a variety of personal, household, or healthcare ingredients in a formulated product composition. A listing of possible personal, household, or health care ingredients is taught in WO 03/101412, which is incorporated herein by reference. Thus, they can be used in wipes, antiperspirants, deodorants, skin creams, skin care lotions, moisturizers, facial treatments such as acne or wrinkle removers, personal and facial cleansers, bath oils, perfumes, colognes, sachets, sunscreens, pre-shave and after-shave lotions, liquid soaps, shaving soaps, shaving lathers, hair shampoos, hair conditioners, hair sprays, mousses, permanents, depilatories, hair cuticle coats, make-ups, color cosmetics, foundations, blushes, lipsticks, lip balms, eyeliners, mascaras, oil removers, color cosmetic removers, nail polishes, and powders.

The water in silicone emulsions of the present invention can also be used to prepare multiple phase emulsions, such as water/silicone/water emulsions, or silicone/water/silicone emulsions, as taught in U.S. Pat. Nos. 5,948,855 and 6,221,927, which are incorporated herein by reference.

The present water in silicone emulsions are particularly useful in sprayable personal care formulations. Such formulations have a maximum viscosity of 2100 mPa·s, but typically range from 1 to 2000 mPa·s, alternatively from 10 to 500 mPa·s.

### EXAMPLES

These examples are intended to illustrate the invention to one of ordinary skill in the art and are should not be interpreted as limiting the scope of the invention set forth in the claims.

### Example 1

Spray-On Skin Moisturizer

| Ingredient | Wt. % | Trade Name/Supplier |
|---|---|---|
| Phase A | | |
| 1. Cyclopentasiloxane and PEG-12 | 20 | Dow Corning ®9011 |
| Dimethicone Crosspolymer | | Silicone Elastomer Blend |
| 2. Cyclopentasiloxane | 9 | Dow Corning ®245 Fluid |
| 3. PPG-3 Miristyl Ether | 1 | Promyristyl PM-3/Croda |
| 4. Essential oils | 3 | TPG 2622/Takasago |
| Phase B | | |
| 5. Distilled Water | 44.5 | |
| 6. Sodium laureth sulfate | 1.5 | Empicol ESB-3/Albright |
| | | and Wilson UK limited |
| 7. Sodium Chloride | 1 | |
| Phase C | | |
| 8. Cyclopentasiloxane | 20 | Dow Corning ®245 Fluid |

Procedure
1. Mix phase A ingredients together
2. Mix phase B ingredients together.
3. Add phase B into phase A very slowly (drop by drop)
4. Mix at 500 rpm during the addition of the first half of water phase and then stir at 800 rpm for the rest of the addition
5. When all water phase is added continue mixing for 15 minutes at 1000 rpm
6. Add phase C and then mix for an additional 10 minutes.

### Example 2

Spray-On Sunny Radiance Formulation

| Commercial name | INCI name | Phase | wt % |
|---|---|---|---|
| DC 9011 Silicone | Cyclopentasiloxane (and) PEG-12 | A1 | 15.00 |
| Elastomer Blend | Dimethicone Crosspolymer | | |
| DC 245 Fluid | Cyclomethicone | A2 | 17.10 |
| Palmotène | | A3 | 0.55 |
| Promyristyl PM3 | PPG-3 Myristyl Ether | A4 | 1.00 |
| Surfmen 448744 | Perfume Symrise | A5 | 0.40 |
| Water | water | B1 | 57.24 |
| DHA | Dihydroxyacetone | B2 | 3.50 |
| Erythrulose | Erythrulose | B3 | 1.50 |
| Sodium chloride | Sodium Chloride | B4 | 0.50 |
| HMW 2220 | Divinyldimethicone/Dimethicone | B5 | 0.50 |
| Non-ionic | Copolymer (and) C12-C13 | | |
| Emulsion | Pareth-23 (and) C12-C13 | | |
| | Pareth-3 | | |
| Empicol ESB 3 | Sodium Laureth Sulfate | B6 | 1.50 |
| FD & C Red 4 1% | Red 4 | B7 | 0.55 |
| in water | | | |
| Citric acid 10% | Citric acid | B8 | 0.16 |
| in water | | | |
| Phenonip | Phenoxyethanol/Methylparaben/ | C | 0.50 |
| | Ethylparaben/Porpylparaben/ | | |
| | Butylparaben | | |
| | | | 100.00 |

Procedure
1) Mix phase A ingredients until homogenous.
2) Dissolve B2 in B1. Add B3-B8 and mix until homogenous.
3) Add phase B quickly to phase A and stir for 10 minutes.
4) Add C and stir for additional 2 minutes.

### Example 3

Spray-On Sun Care Formulation

| | | Quantite |
|---|---|---|
| INCI name | Trade name | % |
| Phase A | | |
| Cyclopentasiloxane & PEG-12 | Dow Corning ® | 20.00 |
| Dimethicone Crosspolymer | 9011 Silicone | |
| | Elastomer | |
| | Blend | |
| Cyclopentasiloxane | Dow Corning ® | 4.50 |
| | 245 Fluid | |
| PPG-3 Miristyl Ether | Promyristyl PM-3 | 1.00 |
| Phenyl Trimethicone | Dow Corning ® | 3.00 |
| | 556 Cosmetic | |
| | Fluid | |
| Polysilicone-15 | Parsol SLX | 7.00 |
| Phase B | | |
| Distilled water | | 44.50 |
| Sodium laureth sulfate | Empicol ESB-3 | 1.50 |
| Sodium Chloride | | 1.00 |
| Phase C | | |
| Cyclopentasiloxane | Dow Corning ® | 14.50 |
| | 245 Fluid | |
| Titanium Dioxide and | Uvinul TiO2 | 3.00 |
| Trimethoxycaprylylsilane | | |
| | TOTAL | 100.00 |

Procedure:
1- Mix phase A ingredients together
2- Mix phase B ingredients together
3- Add slowly phase B into phase A under moderate agitation
4- Homogenize during ~15 mn at 1000 rpm
5- Mix phase C ingredients together
6- Add phase C to phase AB
7- Homogenize during ~15 mn at 1000 rpm

### Example 4

Spray-On Foundation Formulation

| | | Quantite |
|---|---|---|
| INCI name | nom commercial | % |
| Phase A | | |
| Cyclopentasiloxane & PEG-12 | Dow Corning ® 9011 | 20.00 |
| Dimethicone Crosspolymer | Silicone Elastomer | |
| | Blend | |
| PPG-3 Miristyl Ether | Promyristyl PM-3 | 1.00 |
| Phase B | | |
| Cyclopentasiloxane | Dow Corning ® 245 | 9.00 |
| | Fluid | |
| Di;ethicone/Vinyldimethicone | Dow Corning ® 9701 | 2.00 |
| Crosspolymer & Silica | Cosmetic Powder | |
| Phase C | | |
| Distilled water | | 44.50 |
| Sodium laureth sulfate | Empicol ESB-3 | 1.50 |
| Sodium Chloride | | 1.00 |
| Phase D | | |
| Bis-Hydroxyethoxypropyl | Dow Corning 5562 | 12.80 |
| Dimethicone | Fluid | |
| | Iron AS red | 0.50 |
| | Iron AS white | 6.21 |
| | Iron AS Black | 0.25 |
| | Iron AS Yellow | 1.24 |
| | TOTAL | 100.00 |

Procedure:
1- Mix phase A ingredients together
2- Mix phase B ingredients together
3- Add phase B into phase A
4- Mix phase C ingredients together
5- Add slowly phase C into phase AB and stir during ~10 min
6- Mix phase D ingredients together
7- Add phase D into phase ABC and maintain mixing during ~20 mn

### Example 5

W/Si foundation with SPF containing Silicone Elastomer

| Component | Wt % |
|---|---|
| Oil phase | |
| DC 9011 | 15 |
| DC 9046 | 5 |
| DC 9045 | |
| Phyto Squalane | 1 |
| DC 5562 | 7 |
| Parsol MCX | 7 |
| Z cote | 4 |
| Antileukine | 2 |
| Pigment mix | 11 |
| PM3 | |
| Water phase | |
| Glycerine | 3 |
| NaCl | 1 |
| Water | 43.5 |
| SLES | 0.5 |
| Amount | 100 |

Procedure
1. Make up oil phase and mix
2. Add pigment and mix well at 1200 rpm
3. Add Z cote
4. Deair the water phase and add to oil phase and mix for 10 min
Estimated In-vitro SPF of 50.

### Example 6

W/Si Emulsion enriched with vegetable
oil blend for massaging application

| Trade name | INCI name | % | | |
|---|---|---|---|---|
| Phase A | | | | |
| DC 9011 | Cyclopentasiloxane (and) PEG-12 | 20.00 | 13.00 | 2.60 |
| | Dimethicone Crosspolymer | | | |
| DC 245 | Cyclopentasiloxane | 4.50 | 2.80 | 0.13 |
| Promyristyl | PPG-3 Myristyl Ether | 1.00 | 4.83 | 0.05 |
| PM-3 | | | | |
| DC 5562 | Bis-hydroxyethoxy-propyl | 10.00 | 20.00 | 2.00 |
| | Dimethicone | | | |
| DC HY 4009 | Vegetable Oil Blend | 3.00 | 23.40 | 0.70 |
| Phase B | | | | 0.00 |
| Water | Water | 44.50 | | 0.00 |
| Empicol | Sodium Laureth Sulfate (30%) | 1.50 | 10.58 | 0.16 |
| ESB-3 | | | | |
| Sodium | Sodium Chloride | 1.00 | 5.57 | 0.06 |
| Chloride | | | | |
| Phenochem | Phenoxyethanol/Methylparaben/ | 0.30 | 11.75 | 0.04 |
| | Ethylparaben/Porpylparaben/ | | | |
| | Butylparaben | | | |
| Phase C | | | | 0.00 |
| DC 245 | Cyclopentasiloxane | 14.00 | 2.80 | 0.39 |
| | Phase D | | | 0.00 |
| Perfume | | 0.20 | | 0.00 |
| | | 100.00 | | 6.12 |

This formulation produced a light, white, sprayable emulsion, which was stable at room temperature, and at 40° C. after 2 months. Viscosity: 140 cps (mPa · s) (23° C., 50 rpm, spindle 3, 64%).

### Example 7

Spray-on Nylons containing Si-Acrylates

| Commercial name | INCI name | % | | |
|---|---|---|---|---|
| Phase A | | | | |
| DC 9011 | Cyclopentasiloxane | 20.00 | 13.00 | 2.60 |
| | and PEG-12 | | | |
| | Dimethicone Crosspolymer | | | |
| Promyristyl PM3 | PPG-3 Myristyl Ether | 1.00 | 4.83 | 0.05 |
| DC FA 4001 CM | Acrylates/Polytrimethyl- | 24.00 | 62.00 | 14.88 |
| Silicone | siloxymethacrylate | | | |
| Acrylate | Copolymer (and) | | | |
| | cyclopentasiloxane | | | |
| SpectrAl PC 401 | Fumed Alumina | 2.00 | | 0.00 |
| Phase B | | | | 0.00 |
| Water | Water | 25.00 | | 0.00 |
| Empicol ESB-3 | Sodium Laureth Sulfate | 1.50 | 10.58 | 0.16 |
| Sodium Chloride | Sodium Chloride | 1.00 | 5.57 | 0.06 |
| Phase C | | | | 0.00 |
| DC 245 | Cyclopentasiloxane | 5.50 | 2.80 | 0.15 |
| DC 5562 | Bis-hydroxyethoxy-propyl | 12.00 | 20.00 | 2.40 |
| Carbinol | Dimethicone | | | |
| Fluid | | | | |
| Pigments | | | | 0.00 |
| Pigment Blend | AS-EM LCW | 8.00 | | 0.00 |
| | | 100.00 | | 20.30 |

| Pigment Blend: | | % |
|---|---|---|
| White | AS-EM LCW | 75.00 |
| Yellow Iron oxide | AS-EM LCW | 16.68 |
| Red Iron oxide | AS-EM LCW | 6.25 |
| Black Iron oxide | AS-EM LCW | 2.08 |

Procedure:
1. Mix phase A ingredients at 1000 rpm
2. Mix phase B ingredients.
3. Slowly add water phase B in oil phase A, drop by drop at 1000 rpm.
4. Continue mixing 10 minutes at 1500 rpm.
5. Mix phase C and slowly introduce it in A + B
6. Continue mixing 20 minutes at 1500 rpm.

1. A water in silicone emulsion comprising:
a silicone phase containing;
A) an emollient,
B) a silicone elastomer containing a hydrophilic group,
C) an optional organic non-ionic surfactant, and
an aqueous phase containing;
D) an anionic surfactant,
wherein the weight ratio of the polyether functional silicone elastomer B) to the anionic surfactant D) ranges from 60/1 to 1/1.

2. The water in silicone emulsion of claim 1 wherein the emollient A) contains at least 0.1 wt % of a volatile silicone.

3. The water in silicone emulsion of claim 1 wherein the emulsion contains;
1.0 to 90 wt % A)
0.1 to 50 wt % B)
0 to 30 wt % C)
0.01 to 30 wt % D)
and an amount of water such that the sum of the wt % of A), B), C), and D) and water sums to 100 wt %.

4. A method for making a water in silicone emulsion comprising:
I) preparing a silicone phase comprising;
A) an emollient,
B) a silicone elastomer containing a hydrophilic group,
C) an optional organic non-ionic surfactant,
and an aqueous phase containing;
D) an anionic surfactant,
wherein the weight ratio of the polyether functional silicone elastomer B) to the anionic surfactant D) ranges from 60/1 to 1/1, and
II) adding the aqueous phase to the silicone phase with mixing.

5. (canceled)

6. (canceled)

7. (canceled)

8. (canceled)

9. A composition comprising
(I) water in silicone emulsion comprising a silicone phase containing;
A) an emollient,
B) a silicone elastomer containing a hydrophilic group,
C) an optional organic non-ionic surfactant, and
an aqueous phase containing;
D) an anionic surfactant,
wherein the weight ratio of the polyether functional silicone elastomer B) to the anionic surfactant D) ranges from 60/1 to 1/1 and
(II) an ingredient selected from personal, household and health care ingredients.

10. The composition as claimed in claim 9 wherein the viscosity of the composition is 1 to 2000 mPa·s.
